(19) United States
(12) Patent Application Publication
(10) Pub. No.: US 2012/0253433 A1
(43) Pub. Date: Oct. 4, 2012

(54) PHOTOTHERAPY GARMENT/BLANKET

(76) Inventors: Harel D. Rosen, Belle Mead, NJ (US); Shalom Komornik, Toms River, NJ (US); Arye Rosen, Cherry Hill, NJ (US); Daniella Rosen, Cherry HIll, NJ (US)

(21) Appl. No.: 13/073,205

(22) Filed: Mar. 28, 2011

(51) Int. Cl. A61N 5/06

(52) U.S. Cl. 607/91

(57) ABSTRACT

A phototherapy blanket/garment for easy repair and cleaning includes a blanket fitted with hook-and-loop fasteners which hold in place a plurality of light-emitting-device carriers. Power is applied to the light emitting devices by electrical conductors running along the blanket, through polarized connectors, and conductors running along the carriers.

### BACKGROUND

Hyperbilirubinemia (jaundice) is common in infants, and affects, in some degree, up to 50% of full-term infants, and most preterm infants. The jaundice results from an increase in bilirubin production andor a decrease in its excretion due to liver and kidney immaturity. Bilirubin is the end result of chemical reactions involved in the breakdown of haemoglobin molecules. Bilirubin circulates through the blood stream chiefly in unconjugated form, and is processed by catalysis in the liver for conversion into a water-soluble form, which can then be excreted into the intestines as bile. The livers of newborn infants tend to have limited ability to process bilirubin, so infants are prone to accumulation of unconjugated bilirubin, and thus develop jaundice. In most cases, the jaundice is mild, and resolves spontaneously during the first week of life. However, jaundice is potentially dangerous, as high levels of bilirubin are toxic to brain tissue.

While the immaturity of liver cells is the chief cause of jaundice, there may be pathologic causes, which include haemolytic anemia, polycythemia, extravasated blood, and even metabolic disorders. These pathologic causes can create sudden and severe onset of excess bilirubin levels. The goal of medical intervention is to mitigate or curtail the rise in bilirubin levels in the blood, to avoid a toxic accumulation. Approximately 10% of newborns require such intervention.

It is well known that, when infants are exposed to light in the blue region of the spectrum (410 to 490 nanometers or nm), a photochemical reaction takes place in the skin. The photochemical reaction changes unconjugated bilirubin into a more soluble metabolite known as photobilirubin, which is then excreted into the bile. Such phototherapy has proven to be an effective treatment for the vast majority of infants with unconjugated hyperbilirbinemia.

Infant phototherapy for jaundice was generally administered in phototherapy units, as described in U.S. Pat. No. 6,045,575, issued Apr. 4, 2000 in the name of Daniella and Arye Rosen. The effectiveness of these phototherapy units depend, at least in part, on the irradiance delivered by the light source, and the amount of skin exposed to the light. The light delivery systems then in common use in hospital settings fell into two general categories, the first of which involves a crib-like structure for holding the infant, surmounted by banks of fluorescent or halogen lamps, for delivering light in the abovementioned blue region of the spectrum, at the target intensity of 5 to 9W/cm²/nm of bandwidth. This type of phototherapy unit has a number of disadvantages. First, the target light intensity is at a level at which retinal damage is of concern, and consequently the infant must wear protective eye patches. Secondly, to maximize the area exposed to the phototherapy, the infants must be essentially naked; since such infants have difficulty in temperature regulation, they must be maintained in temperature-controlled isolettes during phototherapy. Maintenance in temperature-controlled isolettes, in turn, tends to reduce the availability of human contact. The bulk and cost of the isolettes, in turn, tends to limit the use of this first type of phototherapy unit to hospital environments.

The second type of phototherapy unit which was generally available is the fiberoptic phototherapy blanket. This is a relatively flexible panel-like support for holding the ends of the fibers of one or more fiberoptic cables adjacent to a surface of the blanket, so that light propagating through the optical fibers is directed toward one side of the panel. This phototherapy blanket can be placed on the bottom of a conventional isolette, so that the infant can be illuminated from the bottom, as well as from the top by fluorescent or halogen lamps conventionally disposed. For infants with milder degrees of hyperbilirubinemia, the fiberoptic phototherapy blanket may with some difficulty be used alone, by wrapping the flexible panel about the infant's body, and securing the panel in place. Since the panel is opaque, there is less concern that the light can affect the infant's eyes, which tends to reduce the need for eye protection. If the panel is wrapped about the torso, the child can be dressed over the panel to keep it warm, and thus attains at least some mobility, which allows parental interaction, albeit limited by the umbilical optical fiber cable. Since such phototherapy blanket units are relatively compact, they are more amenable to home use than the more conventional phototherapy “cribs”. The ability to provide home therapy for mild cases of jaundice tends to reduce healthcare costs by eliminating the need for hospitalization in all but severe cases of jaundice.

The Rosen patent describes a flexible phototherapy blanket or garment in which blue light-emitting devices in the form of light-emitting diodes are affixed to the interior or patient-facing side.

Improved phototherapy devices and methods are desired.

### SUMMARY

A phototherapy arrangement according to an aspect of the disclosure comprises a flexible blanket/garment defining a broad side and fitted with first fasteners. The arrangement also comprises an elongated light-emitting-device carrier carrying light-emitting devices and electrical circuits connecting the light-emitting devices to first electrical connectors mounted on the light-emitting-device carrier. The light-emitting devices may emit blue, green, or blue-green light. The light-emitting-device carrier is fitted with second fasteners for mating with the first fasteners of the blanket/garment to hold the light-emitting-device carrier to the broad side of the garment/blanket with light output ports of the light-emitting devices pointing generally in the same direction. An energizing power connector is mounted on the garment/blanket. Second electrical connectors are fastened to the garment/blanket for mating with the first electrical connectors. Electrical conductors extend on the garment/blanket from the power connector to the second electrical connectors, for providing power through the first and second connectors to the light-emitting devices. In a particular embodiment, the first and second fasteners hold the light-emitting-device carrier to the broad side of the garment/blanket with light output ports of the light-emitting devices pointing generally in the direction of the garment/blanket, and the garment/blanket defines light-passing windows or apertures registered with the light output ports of the light-emitting devices. In another embodiment, a spacing arrangement lies adjacent one of the garment/blanket and the light-emitting-device carrier for spacing the light output ports from a user. If the spacing arrangement is transparent, it may overlie light output ports. The spacing arrangement may comprise a bubble. The garment/blanket may define cooling apertures.

A phototherapy arrangement according to an aspect of the invention comprises a substrate, which may be flexible, defining a broad patient-facing surface and having at least one dimension. An elongated flexible light-emitting device carrier defines first and second ends and first and second broad surfaces, and has a length or dimension between the ends which is no greater than the one dimension of the substrate. The arrangement also comprises a set of light emitting devices, each of which defines energization electrodes and a light output port. The light-emitting devices (LEDs) may include solid-state or semiconductor light-emitting diodes andor organic light-emitting diodes (OLEDs). The light-emitting devices are selected to produce light centered at a particular wavelength, which for purposes of jaundice therapy corresponds to the wavelength of blue, green, or blue-green light. A fastening arrangement is coupled to the light-emitting device carrier and to each light emitting device of the set of light-emitting devices, for fastening the light-emitting devices at selected spaced-apart locations along the device carrier, with the light output ports facing away from the device carrier. First and second electrical connectors are affixed to first and second locations, respectively, along the device carrier. The first and second locations may be adjacent the ends of the device carrier. Flexible electrical circuits lie along the device carrier, and are connected to the first and second electrical connectors and to the electrodes of the light-emitting devices, for energizing the light-emitting devices when electrical energization is applied by way of the first and second electrical connectors to the electrodes of the light-emitting devices. The electrical connectors may be polarized. A physical connection arrangement is provided, including portions affixed to the substrate and portions affixed to the light-emitting device carrier, for fastening the light-emitting device carrier to the substrate with the light output ports facing the patient and with the first and second electrical connectors of the light-emitting device carrier adjacent particular locations of the substrate. Electrical mating connectors adapted to mate with the first and second electrical connectors of the light-emitting device carrier are located at the particular locations of the substrate, and are mated with the first and second electrical connectors of the light-emitting device carrier. The connectors may be selected to be of the type which, when mated, are moisture resistant or moistureproof. Flexible electrical circuits lie along the substrate from a main powering location to the particular locations, and make electrical connection to the electrical mating connectors, for applying electrical power from the main powering location to the light emitting devices by way of the flexible electrical circuits of the substrate, the mating connectors, the electrical connectors, and the flexible electrical circuits lying along the light-emitting device carrier. In one embodiment, a set of spacers is located on the patient-facing side of the phototherapy arrangement so as to impose a minimum distance between the patient and the light-emitting devices of the arrangement, which distance is selected to provide substantially uniform illumination from an array of light-emitting devices having particular beamwidths. The spacers may be transparent. The fastening arrangement coupled to the light-emitting device carrier and to each light emitting device of the set of light emitting devices may comprise a set of apertures through the light-emitting device carrier, dimensioned to accommodate the associated light emitting devices, together with fused or soldered connections to energizing conductors.

A phototherapy arrangement according to an aspect of the disclosure comprises a plurality of elongated flexible light-emitting device carriers, each defining first and second ends, first and second broad surfaces, and having a selected length between the ends. The arrangement also comprises a plurality of sets of light emitting organic, solid-state, or semiconductor devices, each of which defines energization electrodes and a light output port. A fastening arrangement is coupled to each of the plurality of light-emitting device carriers and to each light emitting device of the set of light emitting devices, for fastening the light emitting devices of each set of light emitting devices at selected spaced-apart locations along their respective carriers, with the light output ports facing away from the first broad surface of their respective carriers. First and second electrical connectors are affixed adjacent the first and second ends of each of the carriers. Flexible electrical circuits lie along each of the carriers, and are connected to the first and second electrical connectors and to the electrodes of the light-emitting devices, for energizing the light-emitting devices when electrical energization is applied by way of the first and second electrical connectors to the electrodes of the light-emitting devices. The phototherapy arrangement also comprises a flexible garment/blanket substrate that includes at least first and second portions, each defining a length, and also includes a third portion. The first and second portions of the substrate are nominally spaced apart by the selected length, and the third portion of the substrate joins the ends of the first and second portions of the substrate. A physical connection arrangement is provided, including portions affixed to the substrate and portions affixed to the carriers, for fastening the carriers to the substrate with the light output ports facing the user or patient and with the first and second electrical connectors of the carrier adjacent particular locations of the first and second portions of the substrate, respectively. Electrical mating connectors are mounted on the first and second portions of the substrate, and are mated with the first and second electrical connectors of the carriers. Flexible electrical circuits lie along at least the first and second portions of the substrate from a main powering location, and make electrical connection to the electrical mating connectors, for applying electrical power from the main power location to the light emitting devices by way of the flexible electrical circuits of the substrate, the mating connectors, the electrical connectors, and the flexible electrical circuits lying along the carrier. A particular embodiment further comprises a set of spacers adjacent at least some of the light emitting devices, for setting a minimum spacing between the light emitting devices and a user or patient.

A phototherapy garment according to an aspect of the disclosure comprises a plurality of elongated strips of flexible dielectric material, each of the strips of dielectric material defining first and second broad sides and first and second ends. A hook-and-loop fastener half is mounted on the second broad side of each of the strips of flexible dielectric material. At least a first polarized electrical terminal is associated with the first ends of each of the strips of dielectric material, and at least a second polarized electrical terminal is associated with the second ends of each of the strips of dielectric material. A plurality of first electrical conductors extends discontinuously from the first to the second polarized electrical terminals over the first broad side of each of the elongated strips of dielectric material, thereby defining conductor discontinuities. An array of blue, blue-green, or green light emitting solid-state devices is provided. Each of the light emitting solid-state devices defines electrical terminals, and the electrical terminals of the light emitting solid-state devices are electrically connected to ends of the discontinuities of the electrical conductors of the first broad side of each of the elongated strips of dielectric material such that direct voltage applied to, or “across” the first and second polarized terminals energizes the light emitting solid-state devices of the array. A flexible support material is shaped so as to be worn adjacent a patient's body. The flexible support material defines a patient-facing side and an outside. A plurality of mating hook-and-loop fastener halves are affixed to the patient-facing side of the flexible support material for mating with the hook-and-loop fastener halves on the strips of flexible dielectric material. A least third and fourth polarized terminals are provided, which are adapted for mating with the first and second polarized electrical terminals, respectively. The third and fourth polarized terminals are affixed to the patient-facing side of the flexible support material at locations which are adjacent to the first and second polarized terminals, respectively, of the strips of flexible dielectric material when the hook-and-loop fastener half associated with the strip of flexible dielectric material is mated with a hook-and-loop fastener half of the inside of the flexible support material. A plurality of second electrical conductors is associated with the patient-facing side of the flexible support material. The plurality of second electrical conductors extends from an electrical source location to the third) and fourth polarized terminals.

A kit of parts according to an aspect of the disclosure comprises a flexible garment/blanket having an array of patient-facing blue, blue-green, or green-light emitting devices. A battery arrangement is one of affixed to or adapted to be affixed to the garment/blanket. When so affixed, the battery arrangement is capable of providing electrical power to the array. A photoelectric battery charger is adapted for charging the battery arrangement. In a particularly advantageous embodiment, the kit of pars includes a storage case. The case, when open, defines a cavity dimensioned to accommodate an infant and, when closed, provides accommodation for the flexible garment/blanket, the battery arrangement, and the battery charger.

A phototherapy arrangement comprises a garment/blanket fitted on a side with hook-and-loop fasteners. A plurality of light-emitting-device carriers each define first and second broad sides, and are fitted on said second side with mating hook-and-loop fasteners. A plurality of electrically interconnected light-emitting devices is mounted on the first sides of each of the carriers. At least a first type of polarized electrical connector is mounted at a predefined location on each carrier. The first type of polarized electrical connector is electrically interconnected with the light-emitting devices of the carrier. A mating polarized electrical connector is mounted on the garment/blanket at a location which is adjacent the predefined location when the carrier is mounted by the fasteners to the garment/blanket. The mating electrical connector is mated with the corresponding one of the first type of polarized electrical connectors. A power connector is associated with the garment/blanket and electrically interconnected by conductors extending on the garment/blanket to the mating connectors.

### BRIEF DESCRIPTION OF THE DRAWING

FIG. 1A is a simplified perspective or isometric view, partially exploded, illustrating a portion of an apparatus or arrangement according to an aspect of the disclosure including a substrate and a light-emitting device carrier, FIG. 1B is a side elevation view of the structure of FIG. 1A, looking in the direction of section lines 1B-1B, FIG. 1C is a side elevation view of the structure of FIG. 1A, looking in the direction of section lines 1C-1C, FIG. 1D is a side elevation view of the light-emitting device carrier of FIG. 1A with protective strips in place, FIG. 1E is an end elevation view of the structure of FIG. 1D, FIG. 1F is a simplified perspective or isometric view of a solid-state, semiconductor or organic light emitting device showing the light output port and electrical connection electrodes wrapping from the light-emitting-port side onto an adjacent side, FIG. 1G is a simplified perspective or isometric view of the device of FIG. 1F showing the wrapping of the electrodes from the sides onto the bottom, FIG. 1H is an exploded perspective or isometric view of a carrier such as that of FIG. 1B showing the layout of discontinuous strip conductors, and FIG. 1I illustrates a light-emitting-device electrode layout which is an alternative to that of FIG. 1G, and which is dimensioned for bridging the discontinuities;

FIG. 2A is a simplified, partially exploded perspective or isometric view of a light-emitting device carrier according to an aspect of the disclosure, which may be used in the arrangement of FIG. 1A, and FIG. 2B is a cross-sectional elevation view of the carrier of FIG. 2A showing how the various elements are arranged on the carrier;

FIG. 3 is a simplified exploded, cross-sectional representation of another arrangement according to an aspect of the disclosure, in which the light emitting devices are on the opposite side of the substrate from the patient;

FIG. 4A is a perspective or isometric view, partially exploded to reveal the relationship of elements, of a portion of an arrangement according to an aspect of the disclosure, showing the inclusion of cooling apertures, FIG. 4B is similar to FIG. 4A, with the cooling apertures expanded into larger cooling areas, FIG. 4C illustrates details of the routing of energizing wires on a substrate such as those of FIGS. 4A and 4B, and FIG. 4D illustrates details of the arrangement of FIG. 4C;

FIG. 5A is a simplified perspective or isometric view of a light-emitting-device carrier fitted with a transparent spacer, FIG. 5B is a cross-sectional view thereof, FIG. 5C illustrates a closed end of the spacer of FIG. 5A;

FIG. 6A is a representation of a pair of transparent sheets of plastic welded together at particular locations, with through holes in the welded region, FIG. 6B is a cross-section of the arrangement of FIG. 6A after inflation with fluid, FIG. 6C is a plan view of the inflated structure of FIG. 6B, showing the locations of light emitting devices, weld regions, and through apertures, and showing the outlines of the carriers which carry the light emitting devices;

FIG. 7 is an elevation cross-section of a garment/blanket according to an aspect of the disclosure; and

FIG. 8A illustrates a portion of the contents of the case of a kit of parts for jaundice treatment in locations where power is not available, FIG. 8B illustrates a case in which parts can be stored, and which is also dimensioned to accommodate an infant patient, and FIGS. 8C and 8D illustrate the folded and unfolded states, respectively, of a solar panel arrangement which is dimensioned to be accommodated in the case.

### DESCRIPTION

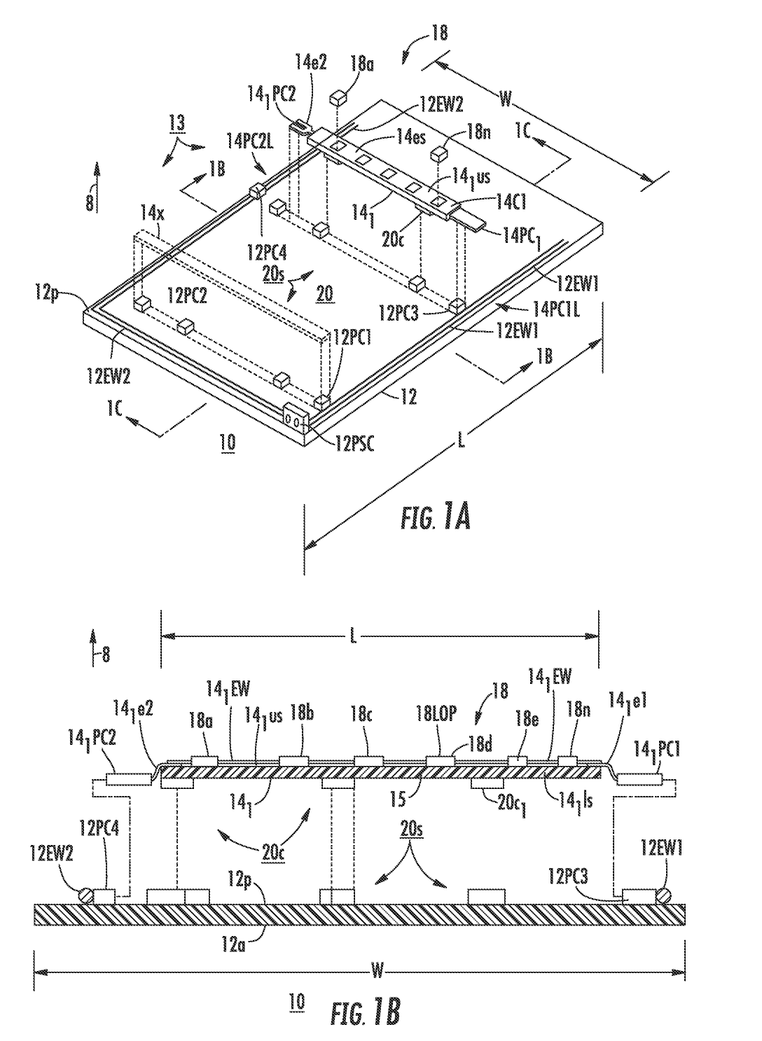

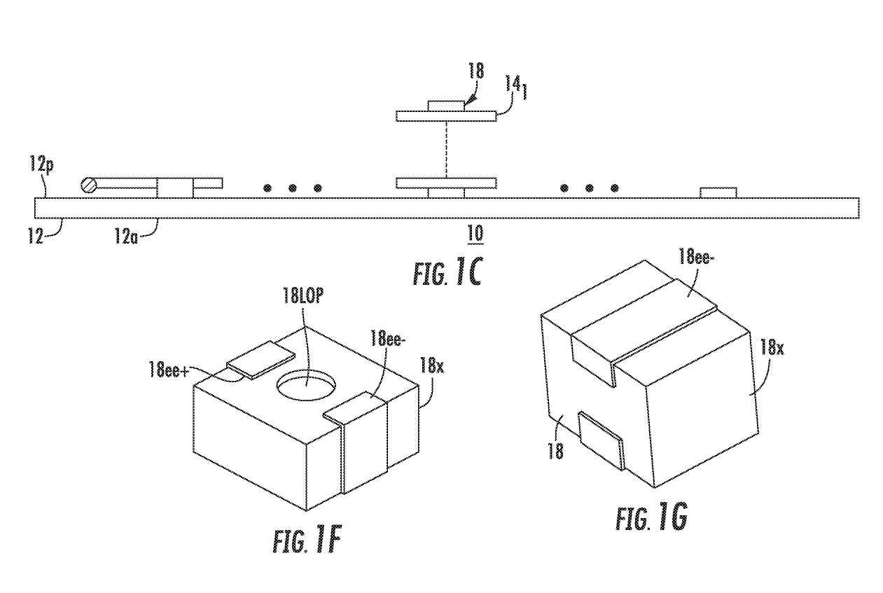

In arrangement 10 of FIGS. 1A, 1B, and 1C, a substrate 12, which may be a flexible substrate such as a blanket or a garment, defines a person- or patient-facing upper side 12p, where the direction to the person or patient is indicated by arrow 8. A plurality or set 13 of elongated light-emitting device (LED) carriers 14 is provided. Only one of the light-emitting device carriers is illustrated, and is designated as 14₁. Another light-emitting device carrier is illustrated in phantom, and is designated 14x. The light-emitting device carrier 14₁ is mounted on the substrate 12 by a set 20 of fastening arrangements. Light-emitting device carrier 14₁ is taken as representative of all the others. Each light-emitting-device carrier of set 13, including light-emitting device carrier 14₁, includes an elongated substrate 15 on which the light-emitting devices are mounted. Side 14₁us of carrier 14₁ faces in the direction of arrow 8. Set 20 of fastening arrangements includes a set of half-fasteners 20S mounted on surface 12p of substrate 12 together with a matching set 20C of corresponding or mating fasteners mounted on a lower surface 14ls of the light emitting device carrier 14₁. The fastener set 20 may be “touch” or “burr” fasteners, also known as “hook-and-loop” fasteners, or may be any other kind of fasteners suited to the use.

Also in FIG. 1A, elongated light-emitting device carrier 14₁ defines first and second ends 14₁e1 and 14₁e2. A polarized electrical connector is mounted at or near each end 14₁e1 and 14₁e2. More particularly, polarized connector 14₁PC1 is mounted at, near, or adjacent to end 14₁e1 of light-emitting device carrier 14₁, and polarized connector 14₁PC2 is mounted at, near, or adjacent to end 14₁e2 of light-emitting device carrier 14₁. Polarized connector 14₁PC1 is of the opposite polarization relative to polarized connector 14₁PC2, which is to say that, if the one is male, the other is female.

The description herein includes relative placement or orientation words such as “top,” “bottom,” “up,” “down,” “lower,” “upper,” “horizontal,” “vertical,” “above,” “below,” as well as derivative terms such as “horizontally,” “downwardly,” and the like. These and other terms should be understood as to refer to the orientation or position then being described, or illustrated in the drawing(s), and not to the orientation or position of the actual element(s) being described or illustrated. These terms are used for convenience in description and understanding, and do not require that the apparatus be constructed or operated in the described position or orientation.

Terms concerning attachments, couplings, and the like, such as “connected,” “attached,” “mounted,” refer in a mechanical context to relationships in which structures are secured or attached to one another either directly or indirectly through intervening structures, as well as both movable and rigid attachments or relationships, unless expressly described otherwise. In an electrical context, such terms have a meaning which includes both direct electrical connection and connection through intermediate electrical elements.

In FIG. 1A, light-emitting device carrier 14₁ of set 13 of light-emitting devices is mounted to the upper or patient-facing surface 12P of substrate 12 by the fastener set 20, with the polarized connectors 14₁PC1 and 14₁PC2 mated with corresponding electrical connectors mounted on or fastened to the substrate 12. The connectors of one pair of the electrical connectors associated with substrate 12 are designated as 12PC1 and 12PC2, and the connectors of another pair are designated 12PC3 and 12PC4. As illustrated in FIG. 1A, connector 14₁PC1 of the light-emitting device carrier 14₁ is located so that it can be mated with corresponding connector 12PC3, and so that connector 14PC2 can be mated with substrate-mounted connector 12PC4. A set of electrical conductors 12EW1 and 12EW2 extend from a polarized power source connector 12PSC to the various surface-mounted connectors 12PC1, 12PC2, 12PC3, 12PC4, . . . . More particularly, negative direct voltage is applied from power source connector 12PSC by way of conductor 12EW1 to polarized connectors 12PC1, 12PC3, . . . and positive direct voltage is applied from power source connector 12PSC by way of conductor 12EW2 to polarized connectors 12PC2, 12PC4, . . . . This provides for powering of as many light-emitting device carriers such as 14 as there are sets of connectors.

Each light-emitting device carrier 14ₓ of set 13 of light-emitting device carriers of FIG. 1A (only carrier 14₁ is shown in FIG. 1A to avoid crowding of the illustration) is mounted in place on or over surface 12P of substrate 12 with a fastener set 20. FIG. 1b is an exploded side elevation view of the arrangement of FIG. 1A, looking in the direction of section lines 1B-1B. In FIG. 1B, elements corresponding to those of FIG. 1A are designated by like reference alphanumerics. As illustrated in FIG. 1B, the light-emitting devices includes devices designated 18a, 18b, 18c, 18d, 18e, . . . , 18n, which are mounted on the upper surface 14₁us of the light-emitting device carrier 14₁. The light-emitting devices may be held in place by adhesive. FIG. 1F is a simplified illustration of a solid-state, semiconductor, or organic light-emitting device 18x (where the subscript x represents any numeric) defining a light-emitting output port 18LOP and a pair of polarized electrical electrodes 18ee+ and 18ee−. The reverse or mounting side 18 mounting of light emitting device 18x is illustrated in FIG. 1G, and shows the two electrodes to which electrical power can be applied. The light output ports LOP of the light-emitting devices of FIG. 1B are located so as to face in the direction of arrow 8. This allows the light produced by the light-emitting devices to illuminate the person or patient. The loop-and-hook fastener halves 20C associated with the bottom surface 14₁ls of the light-emitting device carrier 14₁ lie between the locations of the light-emitting devices of set 18 of light-emitting devices. This positioning of the locations of the fastener halves may be important, depending upon the direction of light emission from the devices of set 18, and the exact mounting configuration of the light-emitting devices. As also illustrated in FIG. 1B, a set 14₁EW of flexible electrical conductors extends along the length of light-emitting device carrier 14₁, and connects each light-emitting device in a series arrangement (or possibly series-parallel) powered from the connectors 14₁PC1 and 14₁PC2.

FIG. 1C is a simplified elevation view of the structure of FIG. 1A looking in the direction of section lines 1C-1C. Elements of FIG. 1C corresponding to those of FIG. 1A are designated by like alphanumerics.

While the light-emitting devices may be sealed against moisture by the manufacturer, it may be desirable to provide additional protection in very moist or wet environments. FIG. 1H is a simplified exploded perspective or isometric view of the arrangement of FIGS. 1A, 1B, and 1C, showing discontinuous electrical conductors, wires, or strip conductors 14EW and designating one of the discontinuities as 14EWD. FIG. 1H also shows the relationship of the discontinuous conductors 14EW to the light-emitting device mounting locations, and FIG. 1I illustrates an alternative metallization pattern on the mounting side of a light-emitting device. FIG. 1D is a side elevation view of a light-emitting device carrier 14₁ similar to that of FIGS. 1A, 1B, and 1C, illustrating a moisture sealing arrangement. The moisture sealing arrangement includes first and second moisture-resistant or moisture-proof plastic strips 30a and 30b, each coated with contact (or other) cement or adhesive 32a, 32b, respectively, on that side facing the light-emitting device carrier 14₁. Since the light leaves output ports 18LOP in the direction of arrow 8, plastic strip 30a and its cement 32a must be transparent (at least over the light output ports), while plastic strip 30b may be opaque if desired. The cement-coated plastic strips 30a and 30b are registered with each other and with the ends 14₁e1 and 14₁e2, and joined, so that the contact cement joins at least the edges of the plastic strips and tends to seal at the exposed electrical wires. FIG. 1E is an end view of the structure of FIG. 1D with the edges of the plastic strips joined and sealed. This leaves exposed only the electrical connectors 14₁PC1 and 14₁PC2, so that moisture ingress to the light-emitting devices and the ancillary circuitry is inhibited. The moisture sealing of the various portions of the apparatus is very advantageous when the user may be neonatal, subject to all the leakages attendant to that state.

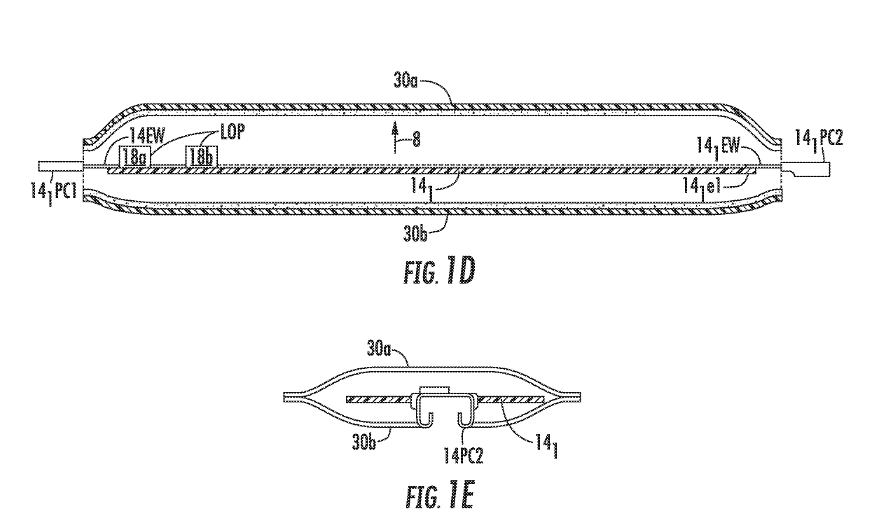

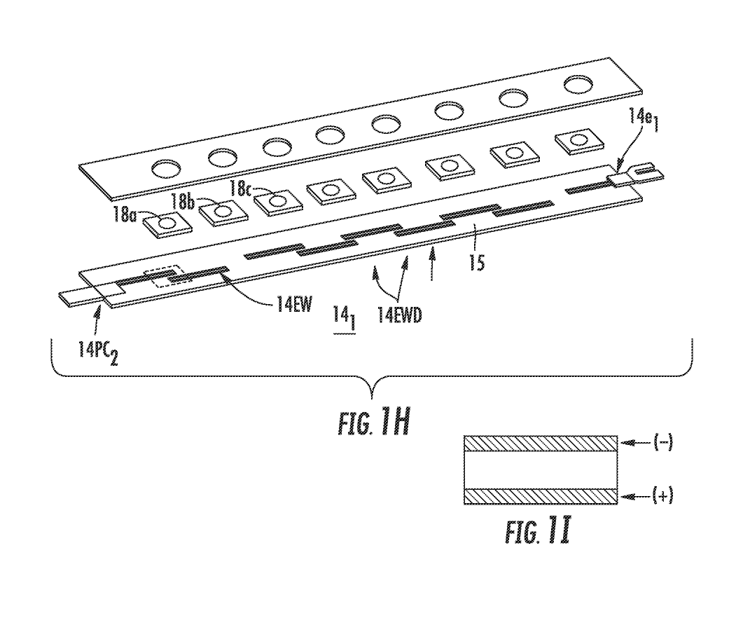

FIG. 2A is a simplified exploded perspective or isometric view illustrating an embodiment of a light-emitting device carrier 210 according to another aspect of the disclosure. In FIG. 2A, a plurality of light-emitting devices 18a, 18b, . . . , 18n are preassembled in a jig (not illustrated) with interconnecting wires or strip conductors 222 and with polarized electrical connectors 224pos and 224neg of a set 224 of connectors to form a flexible arrangement 210. The preassembly may include soldering or fusing the interconnecting wires or strip conductors to the electrodes of the light-emitting devices and to the polarized electrical connectors. As also illustrated in FIG. 2A, a flexible plastic strip 212 is prepared with holes or cutouts 212Ha, 212Hb, . . . , 212Hn spaced apart at the spacing of the light-emitting devices 18 of the assemblage 210, and dimensioned to accommodate the light-emitting devices. The holes or cutouts are provided to allow light to shine through strip 212. The light-emitting devices, such as 18a and 18b, are held in place within the holes or cutouts 212HA and 212Hb by the soldered connection of the electrical contacts at the base of the light-emitting device to the associated electrical wires or strip conductors 222. Cutouts or notches, one of which is illustrated as 212N, may be provided at an end or ends of the plastic strip 212, to provide room for polarized connectors. A layer 212_{cement} of transparent contact cement may be applied to the underside 2121s of strip 212. A further plastic strip 214a, dimensioned to correspond with strip 212, may be coated on its upper side 214aus with a layer of contact cement 214_{contact}. A further flexible, transparent moisture resisting or moistureproof strip 214b is dimensioned to cover strip 212, and is coated on its underside with contact or other cement. Strip 214b is adhesively affixed over strip 212.

FIG. 2B is a simplified longitudinal cross-section of light-emitting device carrier 210 of FIG. 2A. Elements of carrier 210 of FIG. 2B corresponding to those of FIG. 2A are designated by like reference alphanumerics. In FIG. 2B, the protective layers 214a and 214b of plastic strip, each with a layer or film of contact cement or adhesive 214a_{contact} and 214b_{contact}, respectively, are pinched together, generally as described in conjunction with FIGS. 1D and 1E, so as to seal and protect from moisture the interior of the light-emitting device carrier 210. A set 240 of hook-and-loop fastener halves, including halves 240a, 240b, . . . , 240n, is affixed to the upper surface 214bus of plastic strip 240b. These fastener halves are placed at locations between adjacent ones of the light-emitting devices. These fastener halves are for affixing the light-emitting device carrier 210 of FIG. 2A to a substrate or blanket corresponding to 12 of FIG. 1A.

As so far described, the orientation of the light output ports 18LOP of the various light-emitting devices has not been discussed. The light from the light-emitting devices of set 18, as arrayed on the light-emitting device carriers 14 or 210, must reach the intended target, which is the person or patient to be provided with phototherapy. Thus, there can be no opaque regions lying between the light output ports LOP of the light-emitting devices of set 18. In general, the substrate 12 of FIG. 1A may be expected to be opaque, or to at least attenuate light which traverses the “blanket.” For this reason, the light output ports LOP of the various light-emitting devices of set 18 of FIG. 1B are directed away from the substrate 14 and into free space, with only transparent plastic layers between the light output ports and the person or patient, as illustrated in FIGS. 1D and 1E. In

FIGS. 2A and 2B, the light-emitting device carrier strip 210 has hook-and-loop fastener portions affixed to the upper surface 214bus of transparent plastic strip 214b, thereby indicating that the carrier is intended to have its top side (as illustrated in FIG. 2A) fastened to the substrate or blanket 12 of FIG. 1A. For this reason, the light output ports LOP of the various light-emitting devices of set 18 must face downward, namely toward transparent strip 214a of FIG. 2B, rather than upward as illustrated in FIG. 2B.

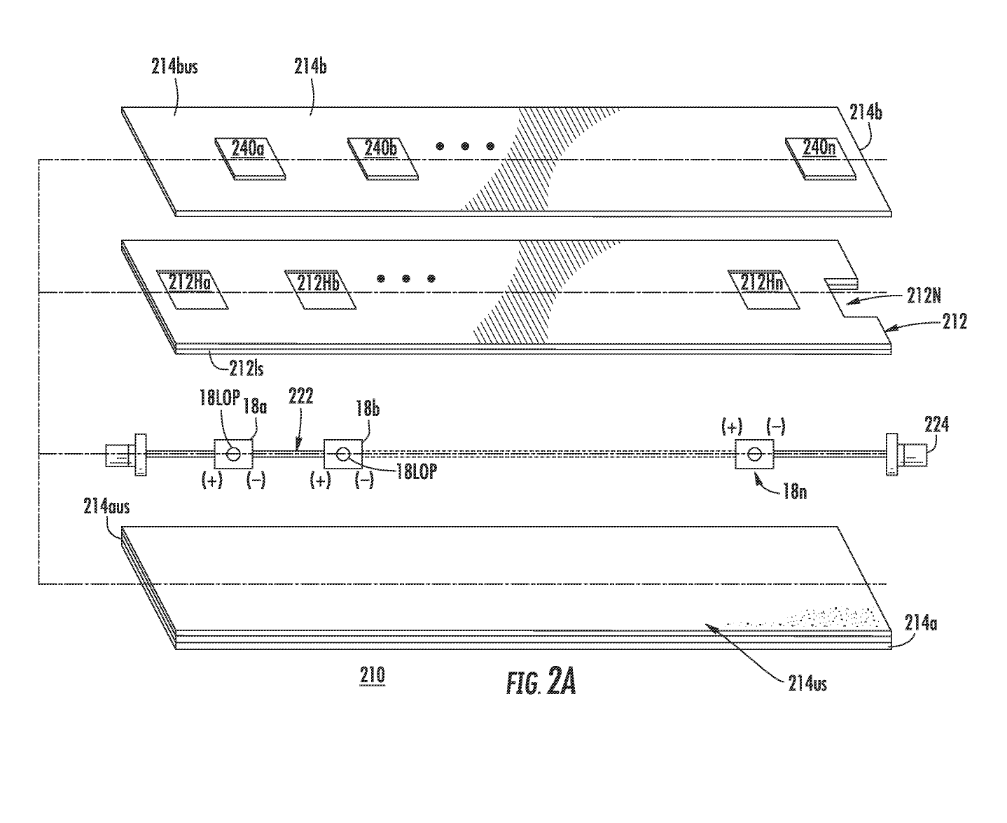

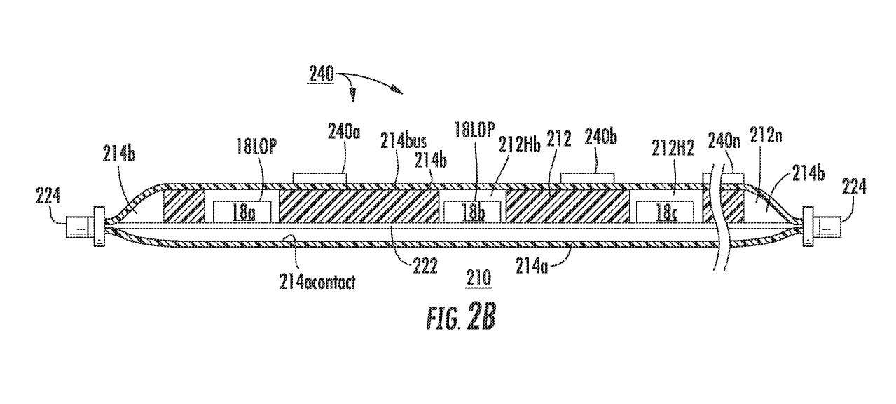

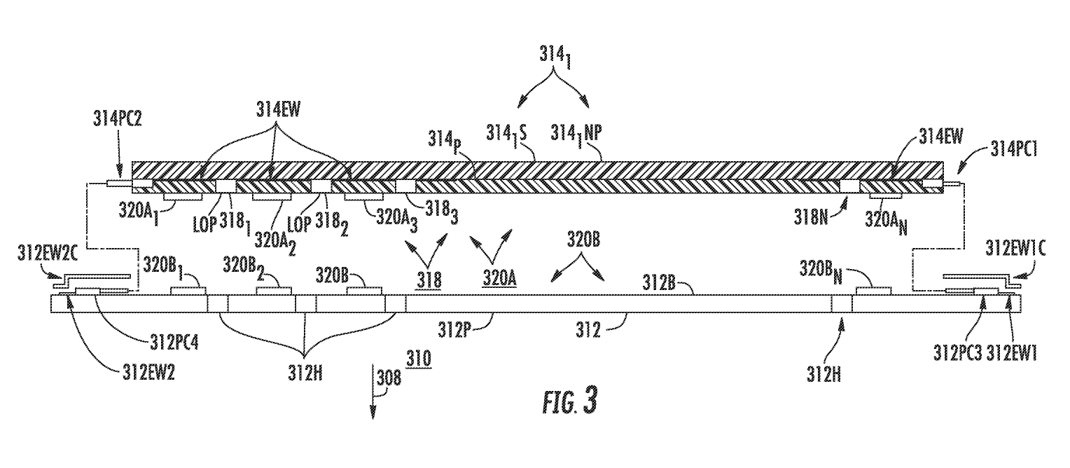

As an alternative to directing the light from the light-emitting devices of each strip light-emitting device carrier away from the substrate, it is possible to define apertures or transparent portions of the substrate at locations registered with the light output ports of the various light-emitting devices of the light-emitting device carrier. FIG. 3 is a simplified cross-section of an arrangement 310 according to an aspect of the disclosure. In FIG. 3, the underlying substrate, blanket or garment is designated 312, and is preferably washable so that sanitary re-use is possible. The direction to the patient or person is illustrated by arrow 308, which identifies the patient-facing surface 312P of substrate 312. Electrical connection wires or conductors lying along the “bottom” surface 312B of the substrate 312 are designated as 312EW1 and 312EW2. Connection wires or conductors 312EW1 and 312EW2 extend perpendicular to the plane of the FIGURE. Conductors 312EW1 and 312EW2 of FIG. 3 perform the same function as conductors 12EW1 and 12EW2 of FIG. 1B, namely the provision of power from a central location (not illustrated) to polarized connectors 312PC3 and 312PC4 and to other polarized connectors. Also illustrated in FIG. 3 is a representative elongated light-emitting-device carrier strip 314₁ of a set of such carrier strips (only one carrier strip illustrated). Light-emitting-device carrier strip 314₁ of FIG. 3 includes two flexible dielectric layers. One of the two flexible dielectric layers is an elongated substrate designated 314₁S, which defines a non-patient-side or surface 314₁NP. The other flexible dielectric layer is a cover designated 314C, which is affixed to the patient-facing side or surface 314P of substrate 314₁S. Light emitting devices 318₁, 318₂, 318₃, . . . , 318_{N} of a set 318 of light emitting devices are affixed to the patient-facing side or surface 314P of substrate 314₁S. More particularly, light emitting devices 314₁, 314₂, 314₃, . . . , 314_{N} are illustrated as having their mounting surfaces 18_{mounting} (see FIG. 1G) mounted on surface 314P of light-emitting device carrier substrate 314₁S. When so mounted, the electrodes of the light-emitting devices lie over the conductor wires or strips 314EW, and are electrically connected thereto in a series or series-parallel manner, well known in the art. Electrical connection wires or conductors lying along the “bottom” surface 314P of the light-emitting-device carrier substrate 314₁S are designated as 314EW, and these conductors perform the same function as conductors 14₁EW of FIG. 1B, namely the provision of power from the polarized connectors of the light-emitting-device carrier to the light-emitting devices. A plurality of fastener halves 320A₁, 320A₂, 320A₃, . . . , 320A_{N} of a set 320A of fastener halves are affixed to the patient-facing side or surface 314CP of cover dielectric sheet 314C at locations between the locations of the light-emitting devices of set 318 of light-emitting devices. More particularly, fastener half 320A₂ is located between light-emitting devices 318₁ and 318₂, and fastener half 320A₃ is located between light-emitting devices 318₂ and 318₃. Fastener halves of set 320A may be either hook or loop portions of hook-and-loop fasteners. A mating set 320B of fastener halves 320B1, 320B2, 320B3, . . . , 320BN is affixed to the “bottom” side 312B of blanket or substrate 312 of FIG. 3, at locations which are registered, when the light-emitting-device carrier 3141 is mounted to blanket substrate 312, with the corresponding fastener halves 320A₁, 320A₂, 320A₃, . . . , 320A_{N} of set 320A of fastener halves. Fastener halves 320A₁, 320A₂, 320A₃, . . . , 320A_{N} of set 320A may mate with fastener halves 320B1, 320B2, 320B3, . . . , 320BN of set 320B. Thus, if fasteners 320A are hook fasteners, fasteners 320B are loop fasteners. Blanket substrate 312 defines a plurality of apertures or holes designated 312H, which are registered with the light output ports (LOP) of the light-emitting devices 318₁, 318₂, 318₃, . . . , 318_{N} of set 318 of light emitting devices when the light-emitting-device carrier 314₁ is mounted on blanket substrate 312. These apertures allow the light produced by the light-emitting devices of set 318 to reach the patient without being absorbed by the blanket.

Thus, the light-emitting devices 318₁, 318₂, 318₃, . . . , 318_{N} of set 318 of light-emitting devices associated with strip carrier 314 of FIG. 3 are spaced apart by a selected dimension (not designated). The spacing in one embodiment is selected to provide ten (10) light-emitting devices on each strip carrier. The spacing is ideally the same for all strip carriers associated with a particular blanket or garment substrate 312. The garment or blanket substrate 312 of FIG. 3 defines transparent windows, apertures or holes, designated 312H in FIG. 3. These windows are spaced apart by the selected distances.

The light-emitting device carrier 314₁ of FIG. 3 has a set 320A of loop-and-hook half-fasteners or half-connectors affixed to the exposed surface of cover strip 314C at locations lying between adjacent ones of the light emitting devices. In FIG. 3, first half-fasteners or half-connectors 320A₁, 320A₂, 320A₃, . . . , 320A_{N} are affixed to the exposed lower surface of cover strip 314C at locations lying between light-emitting devices 318₁, 318₂, 318₃, . . . , Matching “other-half-connectors” 320B₁, 320B₂, 320B₃, . . . , 320Bₙ are mounted or affixed to the upper surface of substrate 312 at locations which (a) do not overlie any of the windows or holes of set 312H, and (b) are registered with the locations of the half-connectors of set 320A. Thus, assuming that the 320A connectors are “loop” type and the 320B connectors are “hook” type, each hook connector 320B lies in registry with a loop connector 320A, and both are clear of windows of set 312H. This allows the light-emitting device carriers to be mounted at any selected position along the blanket or garment substrate 312, and in that position, to find connectors for making the physical connection, and also in that position, to have the light output ports of the light-emitting devices registered with a window in the blanket or garment substrate. The polarized power connectors of the light-emitting-device carrier 3141 are mated with the corresponding connectors of the blanket at the location of the particular carrier. More particularly, carrier power connector 314PC1 of FIG. 3 mates with blanket substrate power connector 312PC3, and carrier power connector 314PC2 mates with blanket substrate power connector 312PC4.

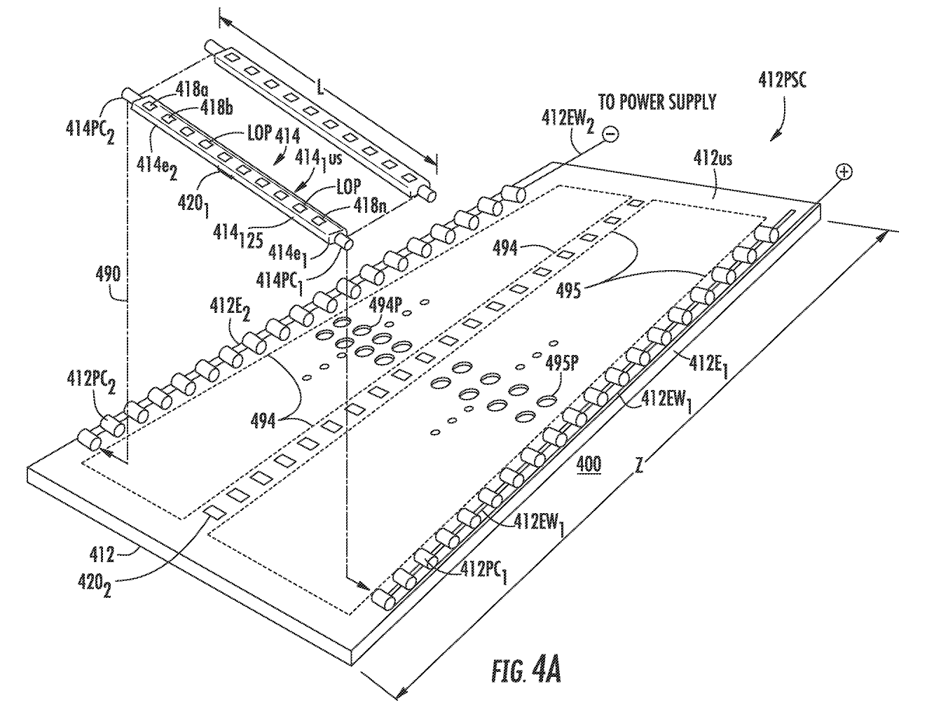

FIG. 4A is an exploded simplified perspective or isometric view of a portion of an arrangement according to an aspect of the disclosure. Elements corresponding to those of FIG. 1A or 1B are designated by like alphanumerics in the 400 series or range. In FIG. 4A, a substrate or “blanket” 412 defines an upper surface 412us. As illustrated in FIG. 4A, a source of direct electrical energy illustrated as a circle with a plus (+) sign is connected to a wire or electrical conductor 412EW1, which is in turn connected to each connector of a given polarization of a set 412PC of polarized connectors, one of which is designated 412PC1. Similarly, the other terminal of the source of direct voltage is illustrated as a circle enclosing a minus (−) is connected to a wire or electrical conductor 412EW2, which in turn is connected to each connector of the other polarization of the set 412PC of connectors. Thus, a single conductor or wire runs along each edge 412E1 and 412E2 of the substrate 412. As also illustrated, a representative light-emitting-device carrier strip 4141 carries ten light-emitting devices, two of which are designated 418a and 418b. The + and − power connectors of carrier strip 414₁ are adapted to be mated with corresponding polarized conductors of set 412PC, as suggested by dot-dash lines 490, 492, and when so mated the carrier strip 414₁ is held to the substrate 412 by loop-and-hook fastener halves such as 420₁. It may be found that the combination of the insulating effect of the blanket substrate, together with the heat associated with the light emitting devices, may tend to overheat the patient, especially in the case of patients with poor temperature regulation. In FIG. 4A, some regions of the blanket or garment substrate is perforated to allow for air flow, to tend to ameliorate temperature rise. The perforated regions in FIG. 4A are delineated by dash lines 494 and 495. Representative perforations in region 494 are illustrated as 494P and in region 495 as 495P. These perforations may be of any shape and any size.

FIG. 4B is similar to FIG. 4A, and corresponding elements are designated by like alphanumerics. FIG. 4B shows the extension of the concept of apertures in regions bounded by lines 494 and 495 of FIG. 4A to complete opening of the regions, as suggested by 494H and 495H. This leaves a central strip of substrate material, designated as 412CS, to which loop-and-hook fastener halves, such as 420₁, may be affixed. Naturally, there may be many gradations between the aperture distributions of FIGS. 4A and 4B, delineating few or many strip supports such as 412CS.

FIG. 4C is a simplified partial representation of a substrate 452 generally similar to the substrate 412 of FIG. 4A. The difference between the arrangements of FIGS. 4A and 4C is that both the + and − electrical interconnection wires in FIG. 4C run along only one side or edge 452E1 of substrate 452. Single polarized connectors can be used to connect each pole to the light-emitting device carrier, so that there are two connectors at an end of each light-emitting device carrier, or a single dual connector can be used at an end of the carrier, as suggested by connector 466.

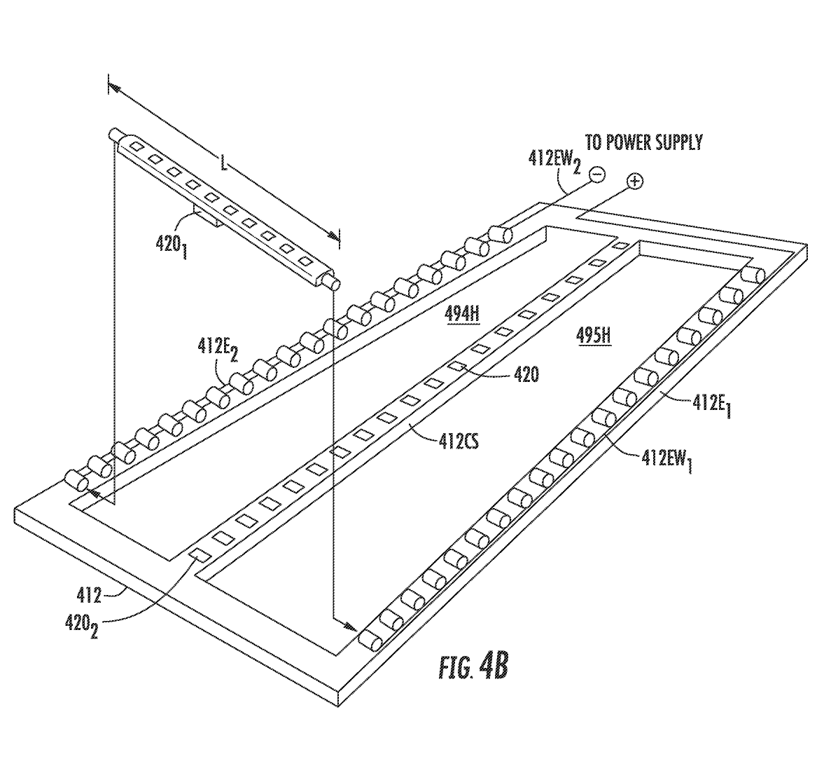

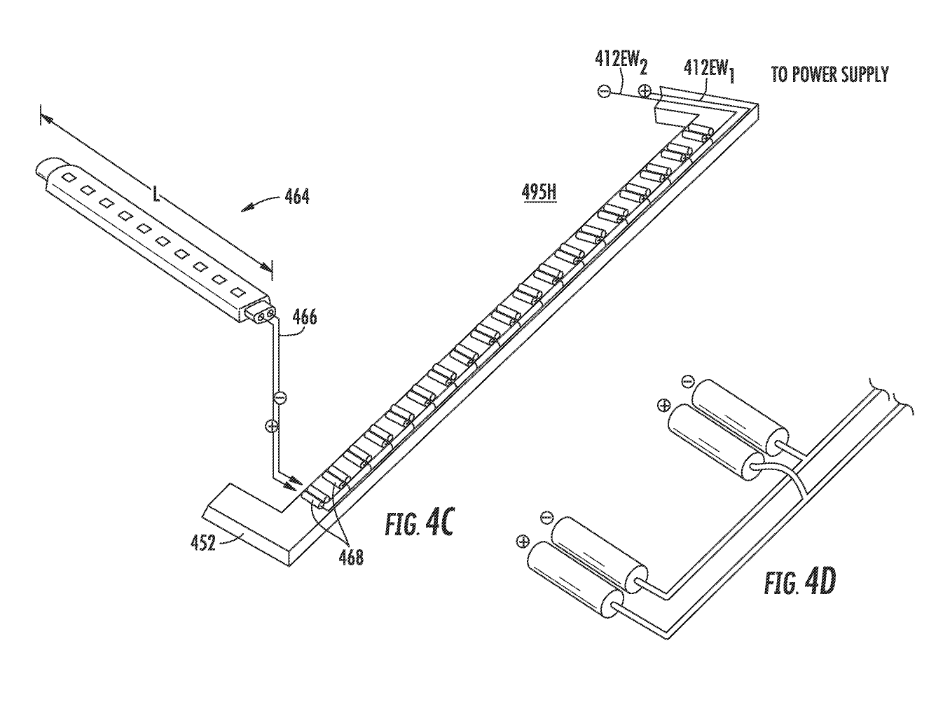

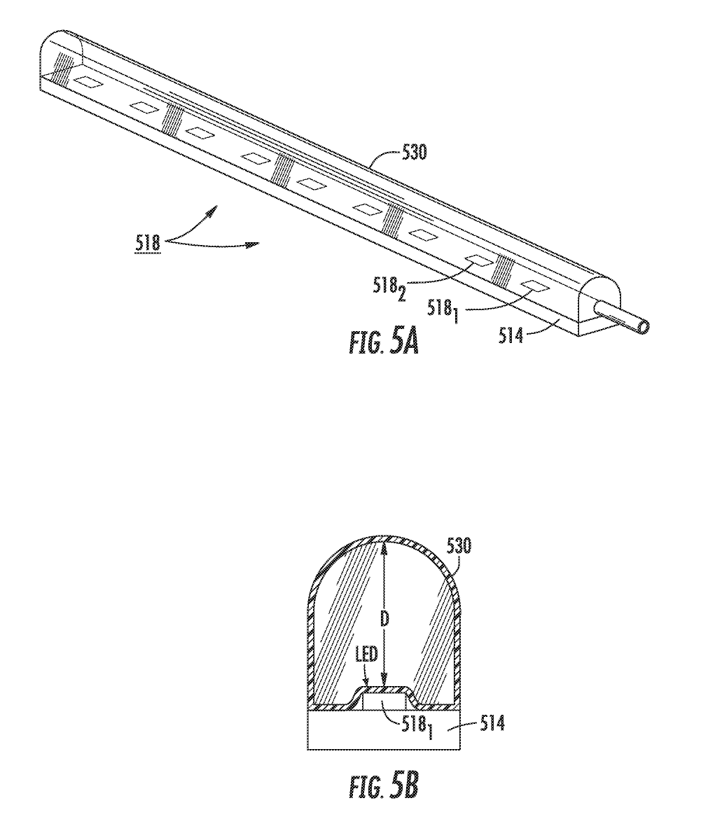

It has been discovered that localized heating can take place at the patient if the beamwidth of the light emitting device is narrow. Also, the distribution of the therapeutic radiation may vary widely across a treatment area, and may be stronger than necessary at locations on the beam axis so as to create heating concerns, and sufficiently weak off-axis so as not to be therapeutically desirable. For this reason, wide-beam light-emitting devices are preferred. Even with wide-beam light emitting devices, the distribution of radiation may not be as even as desired. According to an aspect of the disclosure, spacers are provided to stand the light-emitting devices away from the treatment surface (generally the skin) of the patient. The effect of even small standoff or spacing can be remarkable in effectuating even distribution of the radiation at the treatment surface. According to an aspect of the disclosure, a spacer or standoff is provided between the surface being treated and the light-emitting devices of the therapeutic blanket or garment. FIG. 5A is a perspective or isometric view that notionally illustrates a spacer or standoff which is affixed to a light-emitting carrier such as 141 of FIG. 1A, 210 of FIG. 2A, 3141 of FIG. 3, or 4141 of FIG. 4A. In FIG. 5A, a light-emitting device carrier 514 carries a line of a plurality of light-emitting devices of a set 518 of light-emitting devices. Light-emitting devices 518₁ and 518₂ are designated. FIG. 5B is an end elevation view of the device of FIG. 5A. As illustrated, a transparent “tunnel-” or “Quonset-hut-” shaped spacer element 530 is disposed over the line of light-emitting devices. The spacer 530 prevents the surface being treated (the skin of the patient) from being closer than distance D to any light-emitting device of set 518. This, in turn, tends to render the intensity of electromagnetic radiation more even at the surface being treated. The spacer 530 may be self-supporting or it may be a soft membrane, inflated with air or transparent fluid.

According to a further aspect of the disclosure, the “tunnel” or “Quonset-hut” spacer may be inflated by coolant or water, which has the additional advantage of cooling the light-emitting devices. FIG. 5B illustrates a cross-section of a light-emitting device carrier 514, where the membrane 530 is flexible and defines an elongated enclosed region, and abuts the upper surface of a representative light-emitting device 518₁. If the fluid filling the enclosed region is pressurized, the membrane at the light-emitting device will be pressed against the surface, thereby providing good heat transfer characteristics. FIG. 5C illustrates a sealed end 530E of a tunnel-shaped membrane 530, with an inflation tube or nipple 590 affixed to the seal 530E. The use of a single nipple allows for inflation with air or other gas from a compressed source, but for filling with water from a pressurized source, two separate tubes or nipples should be used to allow air bubbles to be removed.

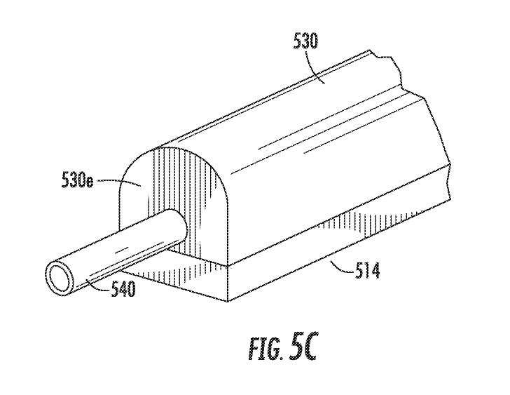

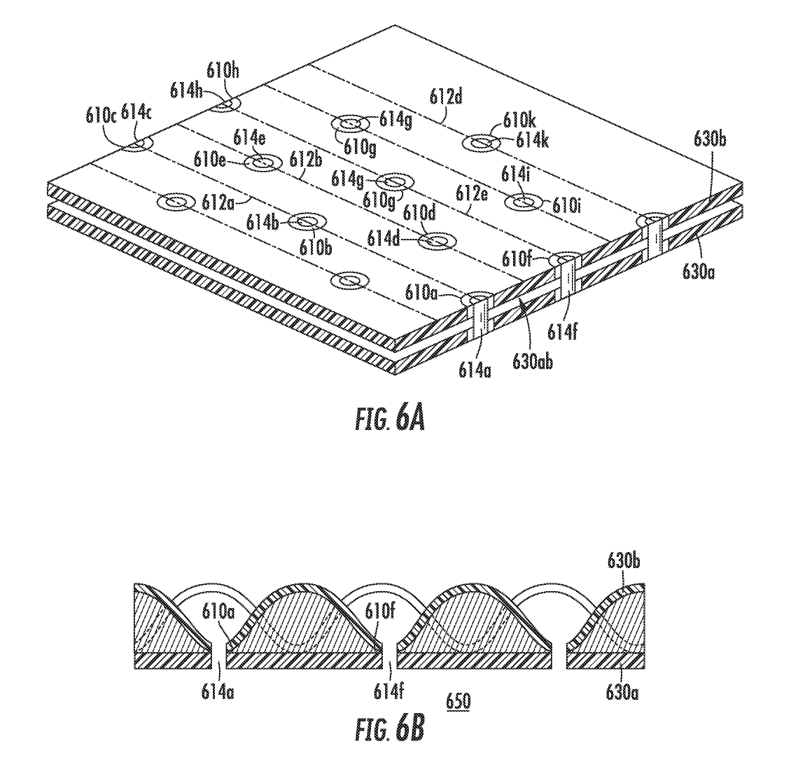

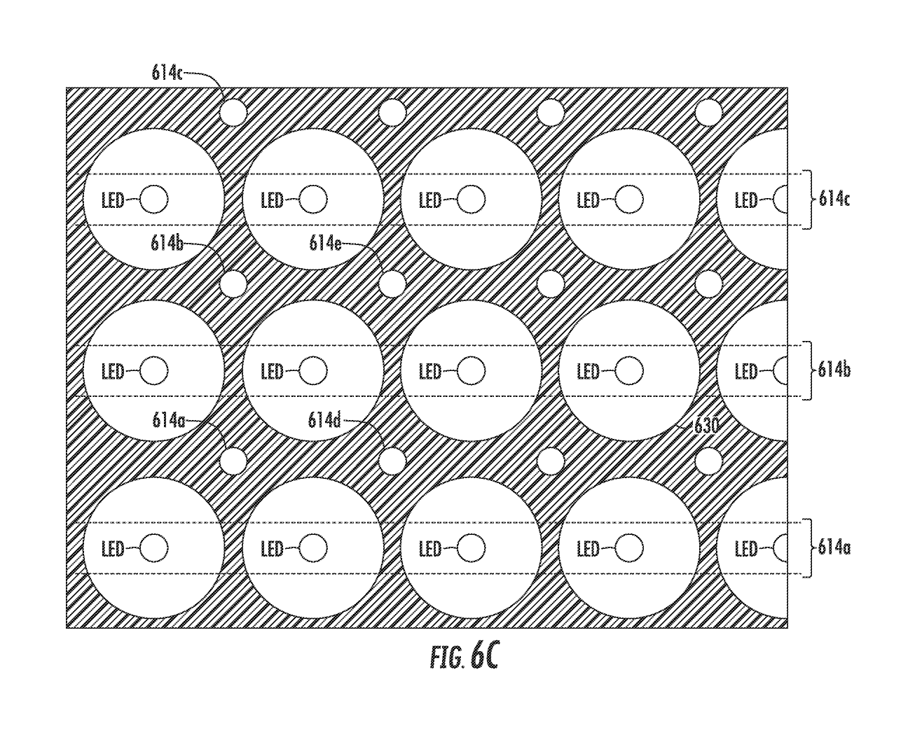

FIG. 6A is a simplified representation of a pair of juxtaposed transparent sheets of plastic. The uppermost sheet is designated 630a, and the lowermost 630b. While juxtaposed, an inflatable space 630aab lies between the sheets. Sheets 630a and 630b are welded or fastened together by an array of annular weld regions 610, and more particularly by weld regions 610a, 610b, 610c, 610d, 610e, 610f, 610g, 610h, 610i, and 610j. In the array, weld regions 610a, 610b, and 610c lie along a straight line 612a, weld regions 610d and 610e lie along a straight line 612b, which is parallel with line 612a. Weld regions 610f, 610g, and 610h lie along a line 612c, parallel with line 612a or 612b. Weld regions 610i and 610j lie along a further parallel line 612d. With these locations of the welds, the weld locations are mutually staggered. Each weld region is centered on a through aperture. More particularly, weld region 610a is centered about a through aperture 614a. Aperture 6114a extends through both sheets 630a and 630b. Weld region 610b is centered on through aperture 614b, weld region 610c is centered on through aperture 614c, weld region 610d is centered on through aperture 614d, weld region 610e is centered on through aperture 610e, weld region 610f is centered on through aperture 614f, weld region 610g is centered on through aperture 614g, weld region 610h is centered on through aperture 614h, weld region 610i is centered on through aperture 614i, weld region 610j is centered on through aperture 614j, and weld region 610k is centered on through aperture 614k.

When the interstice 630ab between welded sheets 630a and 630b is pressurized (by means not illustrated), the welded regions do not allow the sheets to separate. However, the region between welds can expand into intercommunicating “bubbles.” These bubbles are illustrated in cross-section in FIG. 6B. The through apertures 614 provide for cooling air flow through the structure, and the welds prevent the plastic sheets from completely separating. The bubbles also act as spacers to place the light sources at a distance from the patient for making the radiation pattern more even. FIG. 6C is a simplified plan view illustrating a structure similar to FIG. 6B, but not identical thereto. As illustrated in FIG. 6C, the welded regions are hatched, and the bubble regions are not hatched. The locations of the light emitting devices under the peaks of the bubbles are indicated by circles designated LED. The cooling apertures, including apertures 614a, 614b, 614c, 614d, and 614d, lie in the welded region. The locations of the underlying light-emitting-device carriers 614a, 614b, and 614c are indicated by dash lines.

FIG. 7 is a simplified cross-section of a structure 700 including a substrate (blanket or garment) 712, one light-emitting-device carrier 714 of a set of such carriers extending into the page or sheet, each with its light-emitting devices 718a, 718b, . . . registered with the highest point of the bubbles of an inflated bubble structure such as 650 of FIG. 6B. Cooling apertures (if any) in the light-emitting-device carrier 714 are registered with the cooling apertures 614 of the bubble structure 650, and the cooling apertures 614 are also aligned with apertures 794 of substrate 712.

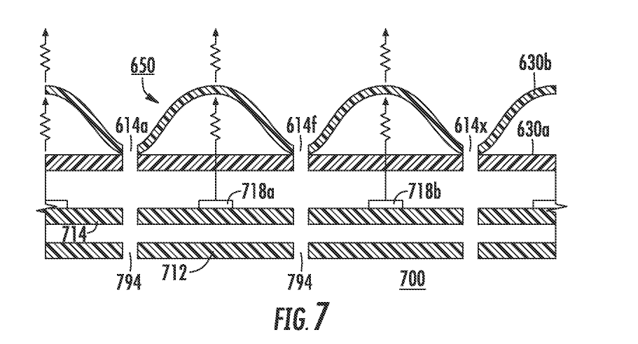

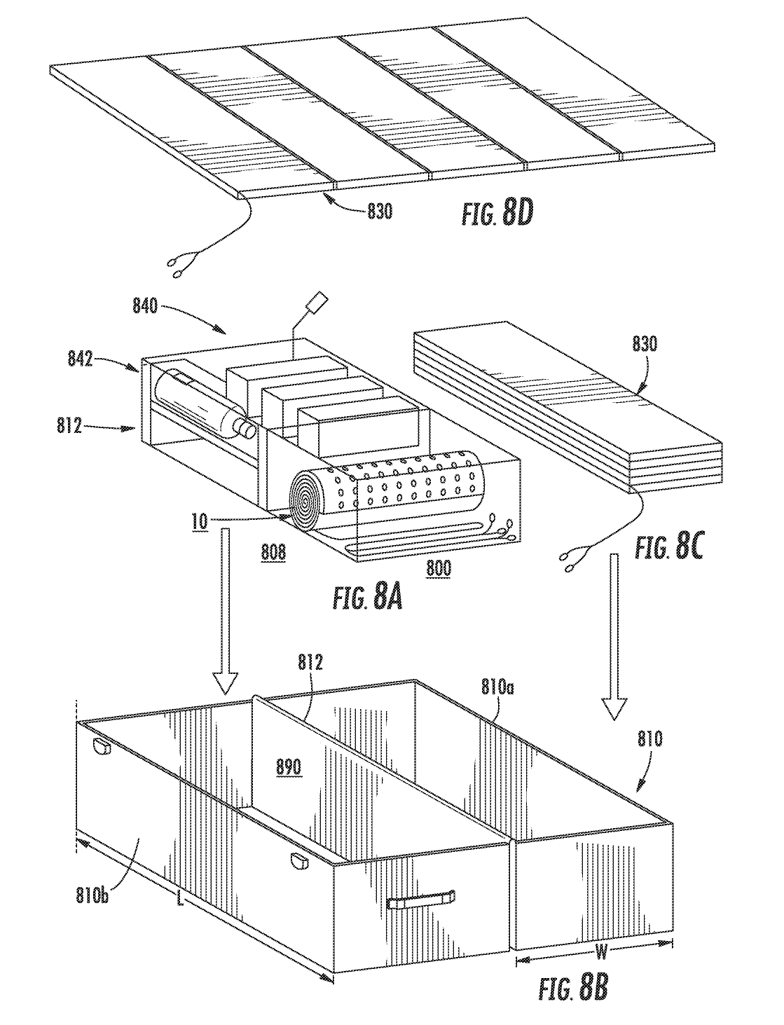

FIGS. 8A, 8B, 8C, and 8D together illustrate a kit of parts useful in providing phototherapy to patients in third-world countries, where both electricity and phototherapy equipment may be scarce. The arrangement of FIG. 8A represents a collection 808 of items which are dimensioned so as to be stored in the case 810 of FIG. 8B. Case 810 includes first and second receptacle portions or “halves” 810a and 810b hinged together at a hinge 812. The dimensions of at least one of the receptacle portions or halves 810a, 810b are selected to accommodate a neonatal child as though it were a crib or bassinet. The dimensions of the folded or flexible/rollable solar panels 830 of FIGS. 8C and 8D are selected to fit into half-case 810a. The collection 808 of items may include a case insert or separator 812, batteries 840, a bilirubin meter 842, and a flexible phototherapy blanket, rolled for storage. The width dimensions of the phototherapy blanket 10 are selected to be slightly less than the width W of the interior of case 810, so that storage can be readily effectuated, and so that a child placed within one of the half-cases 810b can be covered without gaps. The length of the phototherapy blanket in its unrolled state should be slightly less than the length L of the half-case so as not to cover the eyes of a child in the half-case. Ideally, the phototherapy blanket 10 will be fitted with cooling apertures as described in conjunction with FIG. 3, 4A, or 4B and with spacers generally as described in conjunction with FIGS. 6A, 6B, 6C, and 7.

A phototherapy arrangement (10) according to an aspect of the disclosure comprises a flexible blanket/garment (12) defining a broad side (12P) and fitted with first fasteners (20S). The arrangement (10) also comprises an elongated light-emitting-device carrier (14₁) carrying light-emitting devices (18a, 18b) and electrical circuits (14EW) connecting the light-emitting devices (18a, 18b) to first electrical connectors (14₁PC1, 14₁PC2) mounted on the light-emitting-device carrier (14₁). The light-emitting devices (18a, 18b) may emit blue, green, or blue-green light. The light-emitting-device carrier (14₁) is fitted with second fasteners (20C) for mating with the first fasteners (20S) of the blanket/garment (12) to hold the light-emitting-device carrier (14₁) to the broad side (121P) of the garment/blanket with light output ports (LOP) of the light-emitting devices (18a, 18b) pointing generally in the same direction. An energizing power connector (12PSC) is mounted on the garment/blanket (12). Second electrical connectors (12PC3, 12PC4) are fastened to the garment/blanket (12) for mating with the first electrical connectors (14₁PC1, 14₁PC2). Electrical conductors (12EW1, 12EW2) extend on the garment/blanket (12) from the power connector (12PSC) to the second electrical connectors (12PC3, 12PC4), for providing power through the first and second connectors (12PC3, 12PC4) to the light-emitting devices (18a, 18b). In a particular embodiment, the first and second fasteners hold the light-emitting-device carrier (14₁) to the broad side (12P) of the garment/blanket (12) with light output ports (LOP) of the light-emitting devices (18a, 18b) pointing generally in the direction of the garment/blanket (12), and the garment/blanket (12) defines light-passing windows (212) registered with the light output ports (LOP) of the light-emitting devices (18a, 18b). In another embodiment, a spacing arrangement (530, 650) lies adjacent one of the garment/blanket (12) and the light-emitting-device carrier (14₁) for spacing the light output ports (lop) from a user. If the spacing arrangement (530, 650) is transparent, it may overlie light output ports (LOP). The spacing arrangement may comprise a bubble. The garment/blanket (12) may define cooling apertures.

A phototherapy arrangement (10) according to an aspect of the invention comprises a substrate (12), which may be flexible, defining a broad patient-facing surface (12p). The substrate may have at least one dimension (W). An elongated flexible light-emitting device carrier (14) defines first (14e1) and second (14e2) ends and first (14us) and second (14ls) broad surfaces. The light-emitting device carrier may have a dimension between the ends (14e1, 14e2) which is no greater than the one dimension (W) of the substrate (12). The arrangement also comprises a set (18) of light emitting devices (18a, . . . , 18n), each of which defines energization electrodes (18ee+ and 18ee−) and a light output port (18LOP). The light-emitting devices may include solid-state or semiconductor light-emitting devices andor organic light-emitting diodes (OLEDs). The light-emitting devices (18x) are selected to produce light centered at a particular wavelength, which for purposes of jaundice therapy corresponds to the wavelength of blue, green, or blue-green. A fastening arrangement (20) is coupled to the light-emitting device carrier (14a, 14b, . . . , 14x) and to each light emitting device (18) of the set (18) of light-emitting devices, for fastening the light emitting devices (18x) at selected spaced-apart locations along the device carrier (14), with the light output ports (LOP) facing away from the first surface (14us) of the device carrier. First (14PC1) and second (14PC2) electrical connectors are affixed to first (14e1) and second (14e2) locations, respectively, along the device carrier (14). The first and second locations may be adjacent the ends (14e1, 14e2) of the device carrier (14). Flexible electrical circuits (14EW, 222) lie along the device carrier (14), and are connected to the first (14PC1) and second (14PC2) electrical connectors and to the electrodes (18ee+, 18ee−) of the light-emitting devices (18x), for energizing the light-emitting devices (18x) when electrical energization is applied by way of the first (14PC1) and second (14PC2) electrical connectors to the electrodes (18ee+, 18ee−) of the light-emitting devices (18x). The electrical connectors may be polarized. A physical connection arrangement (20) is provided, including portions (20S) affixed to the substrate (12) and portions (20C) affixed to the light-emitting device carrier (14), for fastening the light-emitting device carrier (14) to the substrate (12) with the light output ports (LOP) facing the patient and with the first (14PC1) and second (14PC2) electrical connectors of the light-emitting device carrier (14) adjacent particular locations (of 12PC3 and 12PC4) of the substrate (12). Electrical connectors (12PC3 and 12PC4) adapted to mate with the first (14PC1) and second (14PC2) electrical connectors of the light-emitting device carrier (14) are located at the particular locations of the substrate (12), and mated with the first (14PC1) and second (14PC2) electrical connectors of the light-emitting device carrier (14). The connectors may be selected to be of the type which, when mated, are moisture resistant or moistureproof. Flexible electrical circuits lie along the substrate (12) from a main powering location (12PSC) to the particular locations, and make electrical connection to the electrical mating connectors, for applying electrical power from the main powering location (12PSC) to the light emitting devices (of set 18) by way of the flexible electrical circuits (12EW1, 12EW2) of the substrate (12), the mating connectors (12PC3, 12PC4), the electrical connectors (14PC1, 14PC2), and the flexible electrical circuits (14EW) lying along the light-emitting device carrier (14). In one embodiment, a set of spacers (530, 630) is located on the patient-facing side of the phototherapy arrangement so as to impose a minimum distance between the arrangement and the patient, which distance is selected to provide substantially uniform illumination from an array of light-emitting devices having particular beamwidths. The fastening arrangement (20) coupled to the light-emitting device carrier (14a, 14b, . . . , 14n) and to each light emitting device of the set (14) of light emitting devices may comprise a set of apertures (212H) through the light-emitting device carrier, dimensioned to accommodate the associated light emitting devices.

A phototherapy arrangement (400) according to an aspect of the disclosure comprises a plurality of elongated flexible light-emitting device carriers (set 414), each defining first (414₁e1) and second (414₁e2) ends, first (414₁us) and second (414₁ls) broad surfaces, and having a selected length (L) between the ends (414₁e1, 414₁e2). The arrangement also comprises a plurality of sets (418) of light emitting organic, semiconductor, or solid-state devices (418a, 418b, . . . , 418n), each of which defines energization electrodes (18ee+, 18ee−) and a light output port (LOP). A fastening arrangement (420C) is coupled to each of the plurality of light-emitting device carriers (414₁) and to each light emitting device of the set (418) of light emitting devices, for fastening the light emitting devices of each set (418) of light emitting devices at selected spaced-apart locations along their respective carriers (414), with the light output ports (LOP) facing away from the first broad surface (414₁us) of their respective carriers (414₁)). First (414₁PC1) and second (414₁PC2) electrical connectors are affixed adjacent the first (414₁e1) and second (4141e2) ends of each of the carriers (414). Flexible electrical circuits (14₁EW) lie along each of the carriers (414), and are connected to the first (14₁PC1) and second (14₁PC2) electrical connectors and to the electrodes (18ee+, 18ee−) of the light-emitting devices (418a, 418b, . . . ), for energizing the light-emitting devices (418a, 418b, . . . ) when electrical energization is applied by way of the first (414₁PC1) and second (414₁PC2) electrical connectors to the electrodes (18ee+, 18ee−) of the light-emitting devices (418a, 418b, . . . ). A flexible garment/blanket substrate (412) includes at least first (412e1) and second (412e2) portions, each defining a length (Z), and also includes a third portion (494). The first (412e1) and second (412e2) portions of the substrate (412) are nominally spaced apart by the selected length (L), and the third portion (494) of the substrate (412) joins the ends of the first (412e1) and second (412e2) portions of the substrate (412). A physical connection arrangement (420₁, 420₂) is provided, including portions (420₂) affixed to the substrate (412) and portions (420₁) affixed to the carriers (414), for fastening the carriers (414) to the substrate (412) with the light output ports (LOP) facing the user or patient and with the first (414PC1) and second (414PC2) electrical connectors of the carrier (414) adjacent particular locations of the first (412E1) and second (412E2) portions of the substrate (412), respectively. Electrical mating connectors (412PC1, 412PC2) are mounted on the first (412E1) and second (412E2) portions of the substrate (412), and are mated with the first (414PC1) and second (414PC2) electrical connectors of the carriers (414). Flexible electrical circuits (412EW1, 412EW2) lie along at least the first (412E1) and second (412E2) portions of the substrate (412) from a main powering location (12PSC, 412PSC), and make electrical connection to the electrical mating connectors (412PC1, 412PC2), for applying electrical power from the main power location (412PSC) to the light emitting devices (418) by way of the flexible electrical circuits (412EW1, 412EW2) of the substrate (412), the mating connectors (412PC1, 412PC2), the electrical connectors (414PC1, 414PC2), and the flexible electrical circuits (14EW) lying along the carrier (414). A particular embodiment further comprises a set of spacers adjacent at least some of the light emitting devices, for setting a minimum spacing between the light emitting devices and a user or patient.

A phototherapy garment (10) according to an aspect of the disclosure comprises a plurality of elongated strips of flexible dielectric material (14₁), each of the strips of dielectric material (14₁) defining first (14₁us) and second (14₁ls) broad sides and first (14₁e1) and second (14₁e2) ends. A hook-and-loop fastener half (20C) is mounted on the second broad side (14₁ls) of each of the strips of flexible dielectric material (14₁). At least a first polarized electrical terminal (14₁PC1) is associated with the first ends (14₁e1) of each of the strips (14₁) of dielectric material, and at least a second polarized electrical terminal (14₁PC2) is associated with the second ends (14₁e2) of each of the strips (14₁) of dielectric material. A plurality of first electrical conductors (14EW) extends discontinuously from the first (14₁PC1) to the second (14₁PC2) polarized electrical terminals over the first broad side (14₁us) of each of the elongated strips of dielectric material (14₁), thereby defining conductor discontinuities (14EWD). An array of blue, blue-green, or green light emitting solid-state devices is provided. Each of the light emitting solid-state devices defines electrical terminals, and the electrical terminals of the light emitting solid-state devices are electrically connected to ends of the discontinuities (14EWD) of the electrical conductors (14₁EW) of the first broad side (14₁us) of each of the elongated strips of dielectric material (14₁) such that direct voltage applied to, or “across” the first (14₁PC1) and second (14₁PC2) polarized terminals energizes the light emitting solid-state devices of the array. A flexible support material (12) is shaped so as to be worn adjacent a patient's body. The flexible support material (12) defines a patient-facing side (12P) and an outside (12o). A plurality of mating hook-and-loop fastener halves (20S) are affixed to the patient-facing side (12P) of the flexible support material (12) for mating with the hook-and-loop fastener halves (20C) on the strips of flexible dielectric material (14₁). A least third (12PC3) and fourth (12PC4) polarized terminals are provided, which are adapted for mating with the first (14₁PC1) and second (14₁PC2) polarized electrical terminals, respectively. The third (12PC3) and fourth (12PC4) polarized terminals are affixed to the patient-facing side (12P) of the flexible support material (12) at locations which are adjacent to the first (14₁PC1) and second (14₁PC2) polarized terminals, respectively, of the strips (14) of flexible dielectric material when the hook-and-loop fastener half (20C) associated with the strip of flexible dielectric material (14₁) is mated with a hook-and-loop fastener half (20S) of the inside (12o) of the flexible support material (12). A plurality of second electrical conductors (12EW1, 12EW2) is associated with the patient-facing side (12P) of the flexible support material (12). The plurality of second electrical conductors (12EW1, 12EW2) extends from an electrical source location (12PSC) to the third (12PC3) and fourth (12PC4) polarized terminals.

A kit of parts (800) according to an aspect of the disclosure comprises a flexible garment/blanket (10) having an array of patient-facing blue, blue-green, or green-light emitting devices (18). A battery arrangement (840) is one of affixed to or adapted to be affixed to the garment/blanket (10). When so affixed, the battery arrangement is capable of providing electrical power to the array. A photoelectric battery charger (830) adapted for charging the battery arrangement. In a particularly advantageous embodiment, the kit of pars includes a storage case (810). The case, when open, defining a cavity (890) dimensioned to accommodate an infant and, when closed, provides accommodation for the flexible garment/blanket (10), the battery arrangement (840), and the battery charger (830).

A phototherapy arrangement comprises a garment/blanket (12) fitted on a side (12P) with hook-and-loop fasteners (20S). A plurality of light-emitting-device carriers (14) each define first (14us) and second (14₁ls) broad sides, and are fitted on said second side (14₁ls) with mating hook-and-loop fasteners (20C). A plurality of electrically interconnected light-emitting devices (18a, 18b, . . . ) is mounted on the first sides (14us) of each of the carriers (14). At least a first type of polarized electrical connector (14PC1) is mounted at a predefined location (14₁e1) on each carrier (14). The first type of polarized electrical connector (14PC1) is electrically interconnected with the light-emitting devices (18a, 18b) of the carrier (14₁). A mating polarized electrical connector (12PC3) is mounted on the garment/blanket (12) at a location which is adjacent the predefined location (14₁e1) when the carrier (14) is mounted by the fasteners to the garment/blanket (12). The mating electrical connector (12PC3) is mated with the corresponding one of the first type of polarized electrical connectors (14₁PC1). A power connector (12PSC) is associated with the garment/blanket (12) and electrically interconnected by conductors (12EW1) extending on the garment/blanket (12) to the mating connectors (12PC3).

What is claimed is:

1. A phototherapy arrangement, comprising:
a flexible blanket/garment defining a broad side, said blanket/garment being fitted with fasteners;
an elongated light-emitting-device carrier carrying light-emitting devices and electrical circuits connecting said light-emitting devices to first electrical connectors mounted on said light-emitting-device carrier, said light-emitting-device carrier being fitted with fasteners for mating with said fasteners of said blanket/garment to hold said light-emitting-device carrier to said broad side of said garment/blanket with light output ports of said light-emitting devices pointing generally in the same direction;
an energizing power connector mounted on said garment/blanket;
second electrical connectors fastened to said garment/blanket for mating with said first electrical connectors; and
electrical conductors extending on said garment/blanket from said power connector to said second electrical connectors, for providing power through said first and second connectors to said light-emitting devices.

2. An arrangement according to claim 1, wherein:
said fasteners hold said light-emitting-device carrier to said broad side of said garment/blanket with light output ports of said light-emitting devices pointing generally in the direction of said garment/blanket; and
said garment/blanket defines light-passing windows registered with said light output ports of said light-emitting devices.

3. An arrangement according to claim 1, further comprising a spacing arrangement adjacent one of said garment/blanket and said light-emitting-device carrier for spacing said light output ports from a user.

4. An arrangement according to claim 3, wherein said spacing arrangement is transparent, and overlies said light output ports.

5. An arrangement according to claim 4, wherein said spacing arrangement comprises a bubble.

6. An arrangement according to claim 1, wherein said garment/blanket defines cooling apertures.

7. An arrangement according to claim 1, wherein said light-emitting devices emit blue, green, or blue-green light.

8. A phototherapy arrangement, comprising:
a substrate defining a broad patient-facing surface and having at least one dimension;
an elongated flexible light-emitting device carrier defining first and second ends and first and second broad surfaces, and having a dimension between said ends which is no greater than said one dimension of said substrate;
a set of light emitting devices, each of which defines energization electrodes and a light output port, said light emitting devices of said set of light emitting devices being fastened at selected spaced-apart locations along said first broad surface of said carrier, with said light output ports facing away from said first surface (14us) of said carrier;
first and second electrical connectors affixed to first and second locations, respectively, along said carrier;
flexible electrical circuits lying along said carrier, and connected to said first and second electrical connectors and to said electrodes of said light-emitting devices, for energizing said light-emitting devices when electrical energization is applied by way of said first and second electrical connectors to said electrodes of said light-emitting devices;
a physical connection arrangement including portions affixed to said substrate and portions affixed to said carrier, for fastening said carrier to said substrate with said light output ports facing the patient and with said first and second electrical connectors of said carrier adjacent particular locations of said substrate;
electrical mating connectors mated with said first and second electrical connectors, said mating connectors being located at said particular locations of said substrate; and
flexible electrical circuits lying along said substrate from a main powering location to said particular locations, and making electrical connection to said electrical mating connectors, for applying electrical power from said main power location to said light emitting devices by way of said flexible electrical circuits of said substrate, said mating connectors, said electrical connectors, and said flexible electrical circuits lying along said carrier.

9. An arrangement according to claim 8, wherein said first and second electrical connectors affixed to first and second locations, respectively, along said carrier are polarized electrical connections.

10. An arrangement according to claim 8, wherein said first and second locations along said carrier are at said ends of said carrier.

11. An arrangement according to claim 8, wherein said a set of light emitting devices, includes light-emitting devices which produce light centered about a particular light wavelength.

12. An arrangement according to claim 11,
wherein said particular light wavelength corresponds to one of blue, green, or blue-green.

13. An arrangement according to claim 8, wherein said first and second electrical connectors affixed to first and second locations, respectively, along said carrier, and said electrical mating connectors, are selected to be waterproof when mated.

14. An arrangement according to claim 8, further comprising:
a set of spacers mounted between at least some of said light emitting devices, for setting a minimum spacing between said light emitting devices and the patient.

15. An arrangement according to claim 8, further comprising:
a set of transparent spacers mounted at least partially over said light emitting devices, for setting a minimum spacing between said light emitting devices and the patient.

16. An arrangement according to claim 8, wherein said substrate is flexible.

17. An arrangement according to claim 8, wherein said light emitting devices include at least one of solid-state devices, semiconductor devices, and organic devices.

18. An arrangement according to claim 8, wherein:
said substrate defines at least one dimension; and
said light-emitting device carrier defines a dimension between said ends which is no greater than said one dimension of said substrate.

19. A phototherapy arrangement, comprising:
a plurality of elongated flexible light-emitting device carriers defining first and second ends, first and second broad surfaces, and having a selected length between said ends;
a plurality of sets of light emitting organic, semiconductor, or solid-state devices, each of which defines energization electrodes and a light output port;
a fastening arrangement coupled to each of said plurality of light-emitting device carriers and to each light emitting device of said set of light emitting devices, for fastening said light emitting devices of each set of light emitting devices at selected spaced-apart locations along their respective carriers, with said light output ports facing away from said first surface of their respective carriers;
first and second electrical connectors affixed adjacent said first and second ends of each of said carriers;
flexible electrical circuits lying along each of said carriers, and connected to said first and second electrical connectors and to said electrodes of said light-emitting devices, for energizing said light-emitting devices when electrical energization is applied by way of said first and second electrical connectors to said electrodes of said light-emitting devices;
a flexible substrate including at least first and second portions, each defining a length, and also including a third portion, said first and second portions of said substrate being nominally spaced apart by said selected length, and said third portion of said substrate joining said ends of said first and second portions of said substrate;
a physical connection arrangement including portions affixed to said substrate and portions affixed to said carriers, for fastening said carriers to said substrate with said light output ports facing the patient and with said first and second electrical connectors of said carrier adjacent particular locations of said first and second portions of said substrate, respectively;
electrical mating connectors mounted on said first and second portions of said substrate, and mated with said first and second electrical connectors of said carriers;
flexible electrical circuits lying along at least said first and second portions of said substrate from a main powering location, and making electrical connection to said electrical mating connectors, for applying electrical power from said main power location to said light emitting devices by way of said flexible electrical circuits of said substrate, said mating connectors, said electrical connectors, and said flexible electrical circuits lying along said carrier.

20. An arrangement according to claim 19, further comprising:
a set of spacers mounted between at least some of the light emitting devices, for setting a minimum spacing between said light emitting devices and the patient.

21. A phototherapy garment, comprising:
a plurality of elongated strips of flexible dielectric material, each of said strips of dielectric material defining first and second broad sides and first and second ends;
a hook-and-loop fastener half mounted on said second broad side of each of said strips of flexible dielectric material;
at least a first polarized electrical terminal associated with said first ends of each of said strips of dielectric material;
at least a second polarized electrical terminal associated with said second ends of each of said strips of dielectric material;
a plurality of first electrical conductors extending discontinuously from said first to said second polarized electrical terminals over said first broad side of each of said elongated strips of dielectric material, thereby defining conductor discontinuities;
an array of blue, blue-green, or green light emitting solid-state devices, each of said light emitting solid-state devices defining electrical terminals, said electrical terminals of said light emitting solid-state devices being electrically connected to ends of said discontinuities of said electrical conductors of said first broad side of each of said elongated strips of dielectric material such that direct voltage applied across said first and second polarized terminals energizes said light emitting solid-state devices of said array;
a flexible support material shaped so as to be worn adjacent a patient's body, said flexible support material defining a patient-facing side and an outside;
a plurality of hook-and-loop fastener halves affixed to said patient-facing side of said flexible support material for mating with said hook-and-loop fastener halves on said strips of flexible dielectric material;
a least third and fourth polarized terminals adapted for mating with said first and second polarized electrical terminals, respectively, said third and fourth polarized terminals being affixed to said patient-facing side of said flexible support material at location which are adjacent to said first and second polarized terminals, respectively, of said strips of flexible dielectric material when said hook-and-loop fastener half associated with said strip of flexible dielectric material is mated with a hook-and-loop fastener half of said inside of said flexible support material; and
a plurality of second electrical conductors associated with said patient-facing side of said flexible support material, said plurality of second electrical conductors extending from an electrical source location to said third and fourth polarized terminals.

22. A kit of parts, comprising:
a flexible garment/blanket having an array of patient-facing blue, blue-green, or green-light emitting devices;
a battery arrangement which is one of affixed to or adapted to be affixed to said garment/blanket, and which, when so affixed, is capable of providing electrical power to said array; and
a photoelectric battery charger adapted for charging said battery arrangement.

23. A kit of parts according to claim 22,
further comprising a storage case, said case, when open, defining a cavity dimensioned to accommodate an infant and, when closed, providing accommodation for said flexible garment/blanket, said battery arrangement, and said battery charger.

24. A phototherapy arrangement, comprising:
a garment/blanket fitted on a side with hook-and-loop fasteners;
a plurality of light-emitting-device carriers defining first and second broad sides;
a plurality of electrically interconnected light-emitting devices mounted on said first side of each of said carriers;
at least a first type of polarized electrical connector mounted at a predefined location on each carrier, said first type of polarized electrical connector being electrically interconnected with said light-emitting devices of the carrier;
a mating polarized electrical connector mounted on said garment/blanket at a location which is adjacent said predefined location when said carrier is mounted by said fasteners to said garment/blanket, said mating electrical connector being mated with the corresponding one of said first type of polarized electrical connectors; and
a power connector associated with said garment/blanket and electrically interconnected by conductors extending on said garment/blanket to said mating connectors.
